(19) United States
(12) Patent Application Publication
(10) Pub. No.: US 2025/0239875 A1
(43) Pub. Date: Jul. 24, 2025

(54) RADIATION IMAGING APPARATUS, CHARGING METHOD FOR CHARGING RADIATION IMAGING APPARATUS, AND STORAGE MEDIUM

(71) Applicant: CANON KABUSHIKI KAISHA, Tokyo (JP)

(72) Inventors: TAKASHI YAMAZAKI, Saitama (JP); SHIGERU MAKISHIMA, Tochigi (JP)

(21) Appl. No.: 19/071,312

(22) Filed: Mar. 5, 2025

Related U.S. Application Data
(63) Continuation of application No. PCT/JP2023/031670, filed on Aug. 31, 2023.

(30) Foreign Application Priority Data
Sep. 6, 2022 (JP) 2022-141419

(51) Int. Cl. H02J 7/00; A61B 6/00

(52) U.S. Cl. CPC H02J 7/0071; H02J 7/0047; A61B 6/56

(57) ABSTRACT

A radiation imaging apparatus includes a battery mounting unit on which any type of battery selected from among a plurality of types of batteries is mountable and to and from which the battery is attachable and detachable, an identification unit configured to identify a type of a battery mounted on the battery mounting unit, and a charging unit configured to charge the battery mounted on the battery mounting unit using a charging method according to the type of the battery identified by the identification unit, wherein power is fed from the battery mounted on the battery mounting unit.

### CROSS-REFERENCE TO RELATED APPLICATIONS

This application is a Continuation of International Patent Application No. PCT/JP2023/031670, filed Aug. 31, 2023, which claims the benefit of Japanese Patent Application No. 2022-141419, filed Sep. 6, 2022, both of which are hereby incorporated by reference herein in their entirety.

### BACKGROUND OF THE INVENTION

### Field of the Invention

The present invention relates to a radiation imaging apparatus, a charging method for charging a radiation imaging apparatus, and a storage medium.

### Background Art

In recent years, as a medical radiation imaging apparatus, a portable radiation imaging apparatus on which a battery is mounted is prevalent to handle a wide variety of imaging techniques. As the battery attached to the portable radiation imaging apparatus, a plurality of types of batteries is employed. A lithium-ion capacitor battery, which is excellent in charging/discharging performance, can shorten the charging time, and deteriorates less, or a lithium-ion battery, which is excellent in energy density and has a sufficient capacity even though the battery is small, or a lithium-ion polymer battery is used. These batteries are different in voltage or current when the batteries are charged, and therefore, one of these types of batteries is used in the radiation imaging apparatus.

In contrast, patent literature 1 discusses a technique in which a cradle that charges a radiation imaging apparatus distinguishes the type of a battery built into the radiation imaging apparatus and switches a voltage or a current when the battery is charged.

### CITATION LIST

### Patent Literature

PTL 1: Japanese Patent Laid-Open No. 2012-151994

However, in the method discussed in patent literature 1, a dedicated cradle is required to switch the voltage or the current when the battery is charged. If there is not a dedicated cradle, the charging voltage or the charging current cannot be changed according to the type of the battery.

### SUMMARY OF THE INVENTION

The present invention is directed to enabling the mounting of any type of battery and enabling the charging of a battery using a charging method according to the type of the battery without a dedicated cradle.

A radiation imaging apparatus includes a battery mounting unit on which any of a plurality of types of batteries is mountable and to and from which the mounted battery is attachable and detachable, an identification unit configured to identify a type of a battery mounted on the battery mounting unit, and a charging unit configured to charge the battery mounted on the battery mounting unit using a charging method according to the type of the battery identified by the identification unit, wherein power is fed from the battery mounted on the battery mounting unit.

Further features of the present invention will become apparent from the following description of exemplary embodiments with reference to the attached drawings.

### BRIEF DESCRIPTION OF THE DRAWINGS

FIG. 1 is a diagram illustrating an example of a configuration of a radiation imaging system.

FIG. 2 is a diagram illustrating an example of a configuration of a two-dimensional detector.

FIG. 3 is a timing chart illustrating a control method for controlling a radiation imaging apparatus.

FIG. 4A is a diagram illustrating an example of a structure of a battery attachment unit.

FIG. 4B is a diagram illustrating another example of a structure of a battery attachment unit.

FIG. 4C is a diagram illustrating still another example of a structure of a battery attachment unit.

FIG. 5 is a diagram illustrating an example of a configuration of a charging circuit unit.

FIG. 6 is a diagram illustrating an example of a configuration of a radiation imaging system.

FIG. 7 is a diagram illustrating an example of a configuration of a charging circuit unit.

FIG. 8 is a flowchart illustrating a charging method for charging the radiation imaging apparatus.

FIG. 9A is a diagram illustrating an example of an imaging schedule.

FIG. 9B is a diagram illustrating another example of an imaging schedule.

FIG. 9C is a diagram illustrating still another example of an imaging schedule.

FIG. 9D is a diagram illustrating yet another example of an imaging schedule.

### DESCRIPTION OF THE EMBODIMENTS

Exemplary embodiments will be described below with reference to the drawings. In the following description and the drawings, components common to a plurality of drawings are designated by common signs. Accordingly, the common components are described with reference to the plurality of drawings, and the description of the components designated by the common signs is appropriately omitted. Examples of radiation can include an α-ray, a β-ray, and a γ-ray, which are beams created by particles (including photons) emitted due to radiation decay, and can also include beams having comparable or greater energy, such as an X-ray, a particle ray, and a cosmic ray.

### First Exemplary Embodiment

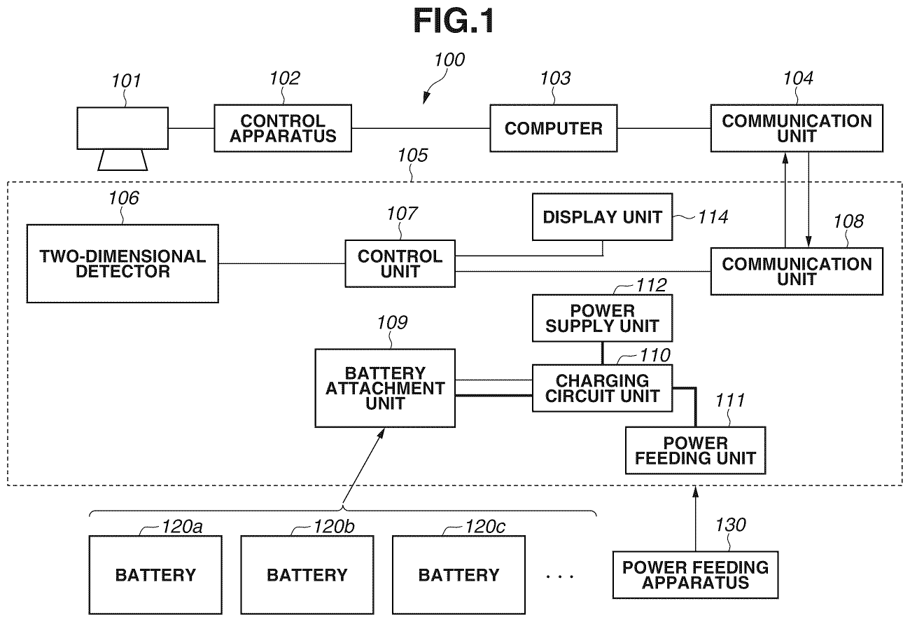

FIG. 1 is a diagram illustrating an example of the configuration of a radiation imaging system 100 according to a first exemplary embodiment. The radiation imaging system 100 includes a radiation generator 101, a control apparatus 102, a computer 103, a communication unit 104, a radiation imaging apparatus 105, and a power feeding apparatus 130.

Under control of the control apparatus 102, the radiation generator 101 delivers radiation. For example, the radiation generator 101 is an X-ray generator that delivers an X-ray. The radiation generator 101 delivers radiation to a two-dimensional detector 106 of the radiation imaging apparatus 105 through an object.

The computer 103 controls the control apparatus 102. The computer 103 also communicates with the radiation imaging apparatus 105 via the communication unit 104, drives the radiation imaging apparatus 105, and acquires an image from the radiation imaging apparatus 105. Wireless communication is suitably used for communication between the communication unit 104 and a communication unit 108 of the radiation imaging apparatus 105, and an access point is suitably used as the communication unit 104. Alternatively, the communication between the communication units 104 and 108 may be communication using a wired local area network (LAN). The communication unit 104 may be built into the computer 103.

The radiation imaging apparatus 105 includes a two-dimensional detector 106, a control unit 107, a communication unit 108, a battery attachment unit 109, a charging circuit unit 110, a power feeding unit 111, a power supply unit 112, and a display unit 114. The two-dimensional detector 106 detects radiation. The control unit 107 controls the operation of the radiation imaging apparatus 105. The radiation imaging apparatus 105 detects radiation and generates image information based on the radiation. For example, the radiation imaging apparatus 105 is an X-ray imaging apparatus that detects an X-ray and generates image information based on the X-ray.

The communication unit 108 communicates with the computer 103 via the communication unit 104. The control unit 107 communicates with the computer 103 via the communication units 104 and 108. The control unit 107 controls the two-dimensional detector 106 using a driving method requested by the computer 103 via the communication units 104 and 108. The control unit 107 also controls the display unit 114.

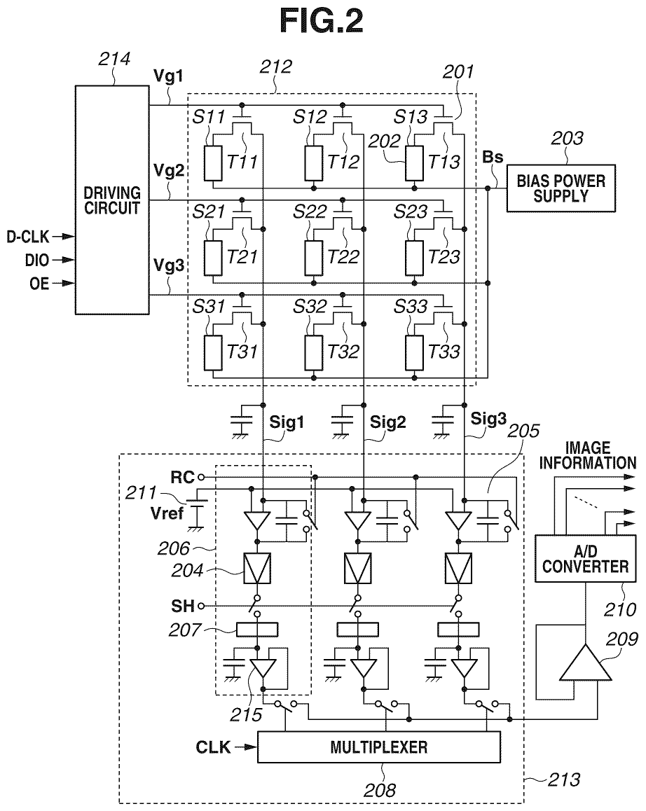

The two-dimensional detector 106 is a sensor in which elements that detect radiation are arranged in an array of X columns and Y rows. The two-dimensional detector 106 detects radiation and outputs image information based on the radiation. The details of the two-dimensional detector 106 will be described below with reference to FIG. 2.

To the battery attachment unit 109, any one of a plurality of types of batteries 120a, 120b, 120c, and so on, attachable to and detachable from the battery attachment unit 109 is attached. In the following description, any of the batteries will be referred to as a “battery 120”. The battery 120 to be attached has a capacity that enables the acquisition of one or more images. Each of the battery 120a, 120b, 120c, and so on, includes a connection portion that is appropriately fitted to the battery attachment unit 109.

The power feeding apparatus 130 feeds power to the radiation imaging apparatus 105. The power feeding apparatus 130 and the radiation imaging apparatus 105 are configured to be attachable to and detachable from each other. If the power feeding apparatus 130 becomes connected to the power feeding unit 111, the power feeding unit 111 feeds power to the charging circuit unit 110. Although the power feeding apparatus 130 is configured to be attachable to and detachable from the radiation imaging apparatus 105, the power feeding apparatus 130 only needs to be configured to feed power to the radiation imaging apparatus 105, and can also be configured to wirelessly feed power.

The charging circuit unit 110 converts a power feeding voltage from the power feeding unit 111 into a voltage according to the type of the battery 120 attached to the battery attachment unit 109 and charges the battery 120 with the voltage according to the type of the battery 120 attached to the battery attachment unit 109. The charging circuit unit 110 also has a current limitation function and limits a charging current to the battery 120 attached to the battery attachment unit 109. The charging circuit unit 110 also converts a power feeding voltage from the power feeding unit 111 into a power supply voltage and supplies the power supply voltage to the power supply unit 112.

The power supply unit 112 feeds power to the components in the radiation imaging apparatus 105. The power supply unit 112 converts a voltage from the charging circuit unit 110 into a predetermined voltage and feeds the predetermined voltage to the two-dimensional detector 106, the control unit 107, the communication unit 108, and the display unit 114. The power supply unit 112 is mainly composed of circuits such as a direct-current-to-direct-current (DC/DC) converter and a series regulator. A circuit where the power supply unit 112 directly receives an input from the charging circuit unit 110 is composed of a DC/DC converter having a step-up/step-down function. Thus, the circuit can excellently feed even various input voltages to a subsequent stage.

As the batteries 120a to 120c, lithium-ion batteries are suitably used, but different types of batteries may be used. For example, each of the batteries 120a to 120c may be a lithium-ion capacitor battery, a lithium polymer battery, an all-solid-state battery, or a nickel-hydrogen battery. As the batteries 120a to 120c, batteries of the same type but different in voltage may be used by changing the number of cells connected in series. Optimal imaging according to the purpose is possible by optionally changing the battery 120.

For example, in use for imaging in an operating room or emergency imaging, there are often few charging opportunities. Thus, it is possible to select a lithium-ion battery or a lithium-ion polymer battery having a large charging capacity. In general imaging in a visiting car or an imaging room, there are many charging opportunities, and the number of images to be captured from the completion of charging to next charging is small. Thus, it is possible to select a lithium-ion capacitor battery having a fast charging speed.

The display unit 114 displays the state of the radiation imaging apparatus 105, thereby notifying a user such as a technologist of the state.

The charging circuit unit 110 recognizes the type of the battery 120 attached to the battery attachment unit 109 and determines a charging method according to the recognized battery 120. This enables the charging circuit unit 110 to charge the battery 120 with a suitable current and a suitable voltage according to the battery 120 attached to the battery attachment unit 109.

Next, an example of the flow of radiation imaging using the radiation imaging system 100 is illustrated. After the user starts the radiation imaging apparatus 105, the user operates the computer 103, thereby bringing the radiation imaging apparatus 105 into the state where imaging can be performed. Next, the user operates the control apparatus 102, thereby setting imaging conditions for delivering radiation (the tube voltage, the tube current, and the delivery time of a radiation tubular lamp). After the above processing ends, the user confirms that imaging is prepared. Then, the user presses a delivery switch included in the control apparatus 102, thereby delivering radiation. In the delivery of the radiation, the control apparatus 102 notifies the radiation imaging apparatus 105 via the computer 103 and the communication unit 104 of a signal indicating that radiation will be delivered from now. In the configuration illustrated in FIG. 1, the radiation imaging apparatus 105 and the control apparatus 102 are connected together via the computer 103 and the communication unit 104, but the connection is not limited to this form.

If the radiation imaging apparatus 105 receives the signal indicating that radiation will be delivered, the radiation imaging apparatus 105 checks whether the delivery of the radiation is prepared. If there is no problem, the radiation imaging apparatus 105 returns delivery permission to the control apparatus 102. Thus, the radiation generator 101 delivers the radiation.

If the radiation imaging apparatus 105 detects the end of the delivery of the radiation using various methods such as receiving a notification from the control apparatus 102 or referring to a setting time determined in advance, the radiation imaging apparatus 105 starts generating image information regarding a radiation image. The generated image information is sent from the radiation imaging apparatus 105 to the computer 103 via the communication unit 104. For example, the image information sent to the computer 103 can be displayed as a radiation image on a display unit (not illustrated) connected to the computer 103. The image is generated from an upper portion to a lower portion, generated from a left portion to a right portion, and displayed in the order of pixels of the generated image information to correspond to the matrix order of pixels read from the two-dimensional detector 106.

FIG. 2 is an equivalent circuit diagram illustrating an example of the configuration of the two-dimensional detector 106 in FIG. 1. FIG. 2 illustrates a detection unit 212 including pixels in 3 rows×3 columns for ease of description. The actual radiation imaging apparatus 105, however, includes more pixels. For example, a 17-inch radiation imaging apparatus 105 includes pixels in about 2800 rows×about 2800 columns.

The two-dimensional detector 106 includes a bias power supply 203, an output buffer amplifier 209, an analog-to-digital (A/D) converter 210, a detection unit 212, a reading circuit 213, and a driving circuit 214.

The detection unit 212 includes a plurality of pixels arranged in a matrix. Each of the plurality of pixels includes a conversion element 202 that converts radiation or light into charges, and a switch element 201 that outputs an electric signal according to the charges. In the present exemplary embodiment, as a photoelectric conversion element that converts light emitted to the conversion element 202 into charges, a metal-insulator-semiconductor (MIS) photodiode placed on an insulating substrate such as a glass substrate and having amorphous silicon as a main material is used, but a PIN photodiode may be used. As the conversion element 202, an indirect conversion element including a wavelength conversion body that converts radiation into light in a wavelength band that can be detected by the photoelectric conversion element or a direct conversion element that directly converts radiation into charges is suitably used on the radiation incidence side of the photoelectric conversion element. As the switch element 201, a transistor including a control terminal and two main terminals is suitably used. In the present exemplary embodiment, a thin-film transistor (TFT) is used.

One of the electrodes of the conversion element 202 is electrically connected to one of the two main terminals of the switch element 201. The other electrode of the conversion element 202 is electrically connected to the bias power supply 203 via a common bias wire Bs. The control terminals of a plurality of switch elements 201 in the row direction (e.g., T11 to T13) are commonly electrically connected to a driving wire Vg1 in the first row. The driving circuit 214 supplies driving signals for controlling the conducting states of the switch elements 201 to the switch elements 201 on a row-by-row basis via the driving wires Vg1 to Vg3.

The other main terminals of a plurality of switch elements 201 in the column direction (e.g., T11 to T31) are electrically connected to a signal wire Sig1 in the first column. The other main terminals of a plurality of switch elements 201 in the column direction (e.g., T12 to T32) are electrically connected to a signal wire Sig2 in the second column. The other main terminals of a plurality of switch elements 201 in the column direction (e.g., T13 to T33) are electrically connected to a signal wire Sig3 in the third column. While the switch elements 201 are in the conducting states, electric signals according to charges in the conversion elements 202 are output to the reading circuit 213 via the signal wires Sig1 to Sig3. The plurality of signal wires Sig1 to Sig3 arranged in the column direction reads electric signals output from the plurality of pixels and transmits the electric signals to the reading circuit 213 in parallel.

In the reading circuit 213, amplification circuits 206 that amplify electric signals output in parallel from the detection unit 212 are provided corresponding to the signal wires Sig1 to Sig3. Each of the amplification circuits 206 includes an integrating amplifier 205 that amplifies an output electric signal, a variable amplifier 204 that amplifies the electric signal from the integrating amplifier 205, a sample hold circuit 207 that samples and holds the amplified electric signal, and a buffer amplifier 215.

The integrating amplifiers 205 include operational amplifiers that amplify electric signals from the signal wires Sig1 to Sig3 and output the amplified electric signals, integrating capacitors, and reset switches. The integrating amplifiers 205 can change the amplification factors by changing the values of the integrating capacitors. To inverting input terminals of the integrating amplifiers 205, electric signals are input from the signal wires Sig1 to Sig3. To non-inverting input terminals of the integrating amplifiers 205, a reference voltage Vref is input from a reference power supply 211. From output terminals of the integrating amplifiers 205, amplified electric signals are output. The integrating capacitors are placed between the inverting input terminals and the output terminals of the integrating amplifiers 205. The sample hold circuits 207 are provided corresponding to the respective amplification circuits 206 and include sampling switches and sampling capacitors.

The reading circuit 213 also includes a multiplexer 208 that sequentially outputs electric signals read in parallel from the amplification circuits 206 as serial image signals to the output buffer amplifier 209. The output buffer amplifier 209 converts the impedances of the image signals and outputs the image signals. The A/D converter 210 converts the image signals as analog electric signals output from the output buffer amplifier 209 into digital image data and outputs the digital image data to the control unit 107 illustrated in FIG. 1.

The reference power supply 211 supplies the reference voltage Vref to the non-inverting input terminals of the integrating amplifiers 205. The bias power supply 203 commonly supplies a bias voltage Vs to the other electrodes of the conversion elements 202 via the bias wire Bs. According to a control signal (D-CLK, OE, or DIO) input from the control unit 107 illustrated in FIG. 1, the driving circuit 214 outputs a driving signal having a conducting voltage Vcom that brings the switch elements 201 into the conducting states and a non-conducting voltage Vss that brings the switch elements 201 into the non-conducting states to the driving wires Vg1 to Vg3. Consequently, the driving circuit 214 controls the conducting states and the non-conducting states of the switch elements 201, thereby driving the detection unit 212.

The control signal D-CLK is a shift clock of a shift register used as the driving circuit 214. The control signal DIO is a pulse transferred from the shift register of the driving circuit 214. The control signal OE is a signal that controls the output end of the shift register of the driving circuit 214.

Based on the above control signals, the time required for and the scanning direction of the driving of the driving circuit 214 are set. The control unit 107 in FIG. 1 provides a control signal RC, a control signal SH, and a control signal CLK to the reading circuit 213, thereby controlling the operations of the components of the reading circuit 213.

The control signal RC controls the operations of the reset switches of the integrating amplifiers 205. The control signal SH controls the operations of the sample hold circuits 207. The control signal CLK controls the operation of the multiplexer 208.

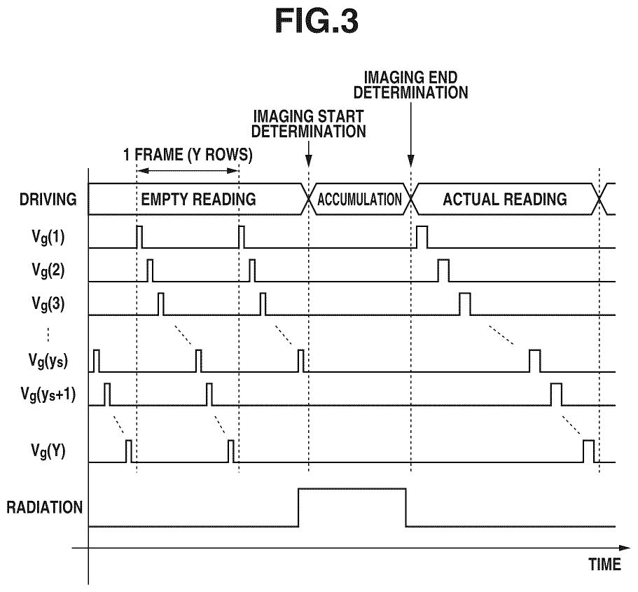

FIG. 3 is a timing chart illustrating an example of the driving timing of the two-dimensional detector 106. Until the delivery of radiation is started, the two-dimensional detector 106 repeats preparation driving for making the switch elements 201 conductive in order from the beginning row (the first row) to the last row (a Y-th row), i.e., empty reading. If the empty reading reaches the last row, the two-dimensional detector 106 returns to the beginning row and continues the empty reading.

If the delivery of the radiation starts, the two-dimensional detector 106 repeats driving for making the switch elements 201 in all the rows non-conductive, i.e., accumulation. Until the delivery of the radiation ends, the two-dimensional detector 106 repeats the accumulation. If the delivery of the radiation ends, the two-dimensional detector 106 performs driving for making the switch elements 201 conductive in order from the beginning row to the last row, reading signals, and performing AD conversion, i.e., actual reading.

Even in the state where no radiation is delivered at all, each pixel of the two-dimensional detector 106 generates a certain signal. This signal is referred to as a “dark current”. The dark current has different characteristics with respect to each pixel. The characteristics of the dark current change according to the temperature of or a change over time in the two-dimensional detector 106. Accordingly, for a signal of each pixel in the capturing of an image, a method for removing the influence of the dark current on an image by obtaining the difference from a signal of the pixel when radiation is not delivered is suitably used. That is, this method individually acquires an image obtained by driving the two-dimensional detector 106 after radiation is delivered and an image obtained by driving the two-dimensional detector 106 without delivering radiation (hereinafter referred to as a “dark image”) and performs a subtraction process on pixels corresponding to each other in these images, thereby obtaining an image of an object.

As described above, to prevent a removal residual error from occurring due to changes in the characteristics themselves of the dark current, it is desirable to acquire a radiation image and a dark image temporally close to each other. Thus, a method for continuously capturing the radiation image and the dark image is suitably used.

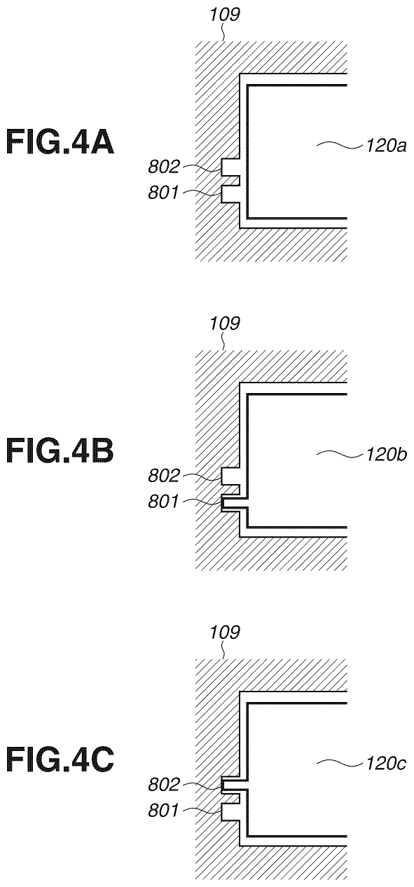

FIGS. 4A to 4C are diagrams illustrating examples of a joining portion between the battery 120 and the battery attachment unit 109 for determining the type of the battery 120. The battery attachment unit 109 includes recessed portions 801 and 802. Each of the recessed portions 801 and 802 is paired with an electrical contact.

FIG. 4A is an enlarged view of a connection surface between the battery attachment unit 109 and the battery 120a. FIG. 4B is an enlarged view of a connection surface between the battery attachment unit 109 and the battery 120b. FIG. 4C is an enlarged view of a connection surface between the battery attachment unit 109 and the battery 120c.

The battery 120b includes a protruding portion that is fitted to the recessed portion 801, and the end of the protruding portion is composed of a conductor. The battery 120c includes a protruding portion that is fitted to the recessed portion 802, and the end of the protruding portion is composed of a conductor. The battery 120a does not include a protruding portion that is fitted to the recessed portion 801 or 802.

FIG. 4A illustrates the state where neither of the recessed portions 801 and 802 is fitted to the battery 120a on the connection surface between the battery attachment unit 109 and the battery 120a. Unlike FIG. 4A, FIG. 4B illustrates the state where the battery 120b includes the protruding portion that is fitted to the recessed portion 801, and the recessed portion 801 is fitted to the protruding portion. FIG. 4C illustrates the state where the battery 120c includes the protruding portion that is fitted to the recessed portion 802, and the recessed portion 802 is fitted to the protruding portion.

The contact paired with the recessed portion 801 or 802 of the battery attachment unit 109 and the conductor of the protruding portion of the battery 120 come into contact with each other, whereby the contact paired with the recessed portion 801 or 802 becomes conductive. The charging circuit unit 110 can determine the type of the battery 120 using an electrical signal based on this conduction.

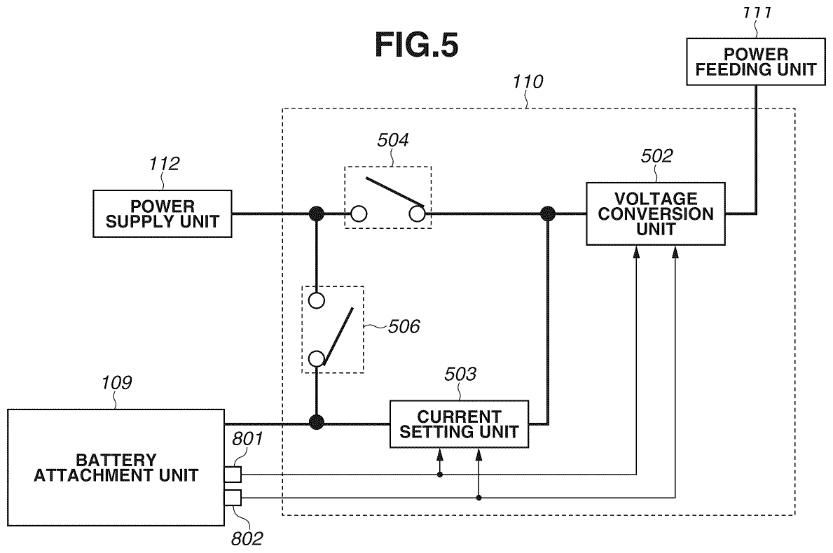

FIG. 5 is a diagram illustrating an example of the configuration of the charging circuit unit 110 in FIG. 1. The charging circuit unit 110 includes a voltage conversion unit 502, a current setting unit 503, and switches 504 and 506. The voltage conversion unit 502 converts a power feeding voltage from the power feeding unit 111 into a voltage set according to the output voltages of the recessed portions 801 and 802 of the battery attachment unit 109. That is, the voltage conversion unit 502 determines the type of the battery 120a in FIG. 4A, the battery 120b in FIG. 4B, or the battery 120c in FIG. 4C according to the output voltages of the recessed portions 801 and 802. Then, the voltage conversion unit 502 converts a power feeding voltage from the power feeding unit 111 into a different voltage according to the type of the battery 120. For example, the voltage conversion unit 502 is composed of a DC/DC converter. The DC/DC converter of the voltage conversion unit 502 is configured to change the voltage division ratio of a feedback resistor by switching a switch. The switch is switched using the output voltages of the recessed portions 801 and 802.

The current setting unit 503 outputs a voltage obtained by conversion by the voltage conversion unit 502 to the battery attachment unit 109 with a current set according to the output voltages of the recessed portions 801 and 802 of the battery attachment unit 109. That is, the current setting unit 503 determines the type of the battery 120a in FIG. 4A, the battery 120b in FIG. 4B, or the battery 120c in FIG. 4C according to the output voltages of the recessed portions 801 and 802. Then, the current setting unit 503 outputs a voltage obtained by conversion by the voltage conversion unit 502 to the battery attachment unit 109 with a different current according to the type of the battery 120. For example, the current setting unit 503 is configured to enable the selection of a plurality of constant current circuits using a switch.

The switches 504 and 506 are selection switches for selecting whether to feed power to the power supply unit 112 from the power feeding unit 111 or feed power to the power supply unit 112 from the battery 120 attached to the battery attachment unit 109. If power is to be fed from the power feeding unit 111, the switch 506 is turned off. If power is not to be fed from the power feeding unit 111, the switch 506 is turned on. If power is to be fed from the power feeding unit 111, the switch 504 is turned on. If power is not to be fed from the power feeding unit 111, the switch 504 is turned off. If power is to be fed from the power feeding unit 111, the switch 504 is turned on, the switch 506 is turned off, and the power feeding unit 111 feeds power to the power supply unit 112 via the voltage conversion unit 502. If power is not to be fed from the power feeding unit 111, the switch 504 is turned off, the switch 506 is turned on, and the battery 120 attached to the battery attachment unit 109 feeds power to the power supply unit 112.

As described above, the charging circuit unit 110 can charge the battery 120 with a current and a voltage according to the type of the battery 120. The battery 120 is charged by performing constant current charging with a current value set in the current setting unit 503 and then performing constant voltage charging with a voltage set in the voltage conversion unit 502. The constant current charging is performed until the voltage in the battery 120 is equal to the voltage set in the voltage conversion unit 502.

The circuit configuration of the voltage conversion unit 502 is not limited to the above configuration, and the voltage conversion unit 502 only needs to be able to change its output voltage according to the output voltages of the recessed portions 801 and 802 of the battery attachment unit 109. For example, the voltage conversion unit 502 may use a method for preparing a plurality of power supply circuits such as a DC/DC converter and a series regulator, preparing analog switches for the inputs and outputs of the power supply circuits, and selecting a power supply circuit itself from the power supply circuits.

The circuit configuration of the current setting unit 503 is not limited to the above configuration, either, and the current setting unit 503 only needs to be able to change its output current according to the output voltages of the recessed portions 801 and 802 of the battery attachment unit 109. For example, the current setting unit 503 may use a method for preparing a plurality of current detection resistors and enabling the selection of a current detection resistor from the current detection resistors using an analog switch.

As described above, in the radiation imaging apparatus 105, the batteries 120a to 120c can be selectively attached to the battery attachment unit 109, and the radiation imaging apparatus 105 can charge the attached battery 120 with a current and a voltage according to the type of the attached battery 120. The radiation imaging apparatus 105 can determine the type of the attached battery 120 and charge the attached battery 120 with an appropriate current and an appropriate voltage.

Although in the present exemplary embodiment, a configuration has been employed in which signals from the recessed portions 801 and 802 of the battery attachment unit 109 are input to the voltage conversion unit 502 as they are, the present invention is not limited to this. The numbers of the recessed portions 801 and 802 can be increased according to the type of the battery 120 to be distinguished. The charging circuit unit 110 may use signals from the recessed portions 801 and 802 by converting the signals. For example, the charging circuit unit 110 may convert two signals output from the recessed portions 801 and 802 as two bits into a four-valued signal indicating “00”, “01”, “10”, or “11”. Although each of the recessed portions 801 and 802 is composed of the conductor paired with the recessed portion, the present invention is not limited to this. The recessed portions 801 and 802 may output signals according to the batteries 120a to 120c. For example, a configuration may be employed in which a switch is pressed by the protruding portion of the battery 120. Alternatively, a configuration may be employed in which each of the recessed portions 801 and 802 has a photointerrupter that operates in the protruding portion of the battery 120 built-in.

Although in the present exemplary embodiment, a description has been given on the assumption that the recessed portions 801 and 802 of the battery attachment unit 109 have functions independent of each other, the present invention is not limited to this. The battery 120 may include a joining portion for inputting and outputting power to and from the charging circuit unit 110. In the battery attachment unit 109, a joining portion with the battery 120 and the charging circuit unit 110 may have a function for determining the type of the battery 120, instead of the recessed portions 801 and 802. Specifically, the battery attachment unit 109 may apply a signal as a substitute for the recessed portions 801 and 802 to a connector used in the joining portion. In the battery attachment unit 109, the joining portion has the functions of the recessed portions 801 and 802, whereby it is not necessary to provide a recessed portion for determining the type of the battery 120 in the battery attachment unit 109, and it is possible to miniaturize the radiation imaging apparatus 105.

As described above, the battery attachment unit 109 is a battery mounting unit. Any type of battery selected from among a plurality of types of batteries can be mounted on the battery attachment unit 109, and the battery can be attached to and detached from the battery attachment unit 109. The charging circuit unit 110 is an identification unit and identifies the type of the battery mounted on the battery attachment unit 109. The charging circuit unit 110 is a charging unit and charges the battery mounted on the battery attachment unit 109 using a charging method according to the identified type of the battery. To the power supply unit 112, power is fed from the battery mounted on the battery attachment unit 109.

Specifically, the charging circuit unit 110 charges the battery mounted on the battery attachment unit 109 with a current or a voltage according to the identified type of the battery. The charging circuit unit 110 identifies the type of the battery mounted on the battery attachment unit 109 according to the shape of the battery mounted on the battery attachment unit 109. In a joining portion with the battery mounted on the battery attachment unit 109, the charging circuit unit 110 may generate a signal for identifying the type of the battery mounted on the battery attachment unit 109.

The charging circuit unit 110 performs constant current charging on the battery mounted on the battery attachment unit 109 with the current according to the identified type of the battery. Then, the charging circuit unit 110 performs constant voltage charging on the battery mounted on the battery attachment unit 109 with the voltage according to the identified type of the battery.

The current setting unit 503 is a current control unit and charges the battery mounted on the battery attachment unit 109 with the current according to the identified type of the battery. The voltage conversion unit 502 is a voltage control unit and charges the battery mounted on the battery attachment unit 109 with the voltage according to the identified type of the battery.

As described above, according to the present exemplary embodiment, in the radiation imaging apparatus 105, the user of the radiation imaging apparatus 105 can select the type of a battery according to the use. The radiation imaging apparatus 105 can appropriately switch a charging current or a charging voltage according to the type of the battery without a dedicated cradle.

### Second Exemplary Embodiment

Next, a second exemplary embodiment is described. The radiation imaging apparatus 105 according to the second exemplary embodiment has imaging schedule information, determines whether the attached battery 120 is appropriate, and determines a charging method according to the amount of charge remaining in the battery 120. This enables the radiation imaging apparatus 105 to perform charging in which the deterioration of the battery 120 is reduced, according to the battery 120 attached to the battery attachment unit 109. Descriptions similar to the first exemplary embodiment are omitted below.

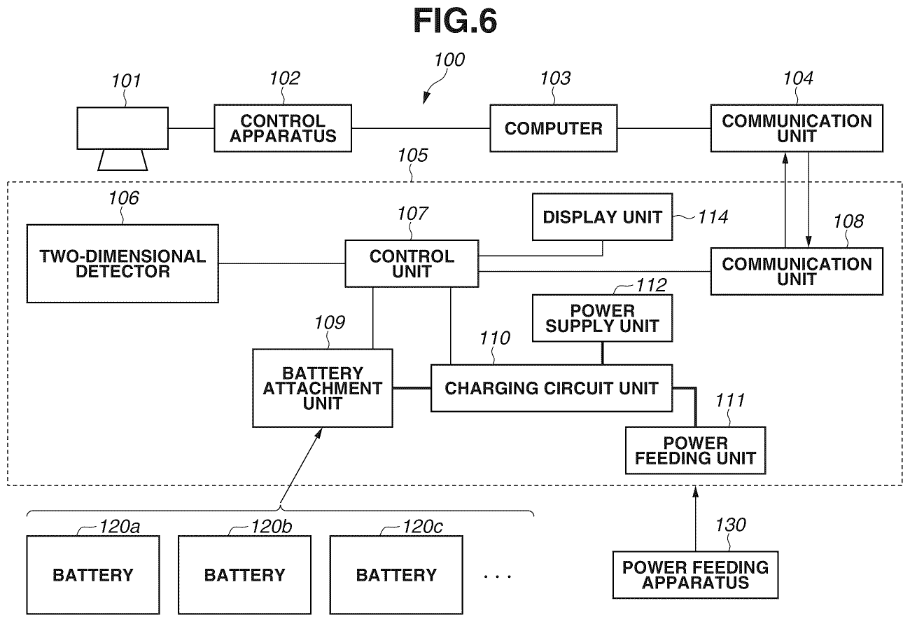

FIG. 6 is a diagram illustrating an example of the configuration of the radiation imaging system 100 according to the second exemplary embodiment. FIG. 6 is different from FIG. 1 in that a control line is provided between the control unit 107 and the battery attachment unit 109, a control line is provided between the control unit 107 and the charging circuit unit 110, and a control line is not provided between the battery attachment unit 109 and the charging circuit unit 110.

The computer 103 in FIG. 6 is different from the computer 103 in FIG. 1 in that the computer 103 in FIG. 6 has imaging schedule information obtained from a radiology information system (RIS) (not illustrated).

The control unit 107 controls the charging circuit unit 110 according to the type of the battery 120 attached to the battery attachment unit 109. The control unit 107 can recognize the attachment of the battery 120 to the battery attachment unit 109 and recognize the type of the attached battery 120. The control unit 107 has a setting voltage value and a setting current value with respect to each type of the battery 120 and a conversion formula for converting from the remaining amount of the battery into the number of images that can be captured as table information. The control unit 107 determines the charging voltage and the charging current of the charging circuit unit 110 according to the recognized type of the battery 120. For example, if the battery 120a becomes attached to the battery attachment unit 109, the control unit 107 identifies the type of the battery 120a attached to the battery attachment unit 109 according to the output voltages of the recessed portions 801 and 802 of the battery attachment unit 109.

The control unit 107 receives the imaging schedule information from the computer 103 via the communication units 104 and 108. The control unit 107 checks the identified type of the battery 120 and the imaging schedule information against each other and displays information indicating whether an appropriate battery 120 is attached on the display unit 114.

Next, the relationship between the imaging schedule information and an appropriate type of the battery 120 is described. For example, in use for imaging in an operating room or emergency imaging, there are often few charging opportunities. Thus, it is desirable to attach a lithium-ion battery or a lithium-ion polymer battery having a large charging capacity. In general imaging in a visiting car or an imaging room, there are many charging opportunities, and the number of images to be captured from the completion of charging to next charging is small. Thus, it is desirable to attach a lithium-ion capacitor battery having a fast charging speed.

For example, a case is described where the battery 120a is a lithium-ion battery and the battery 120b is a lithium-ion capacitor battery. In a case where the radiation imaging apparatus 105 is scheduled to perform imaging in an operating room and the battery 120a is attached to the battery attachment unit 109, the control unit 107 performs display indicating that an appropriate battery is attached on the display unit 114. If, however, the battery 120b is attached to the battery attachment unit 109, the control unit 107 performs display indicating that an appropriate battery is not attached on the display unit 114. Then, the control unit 107 displays an appropriate type of the battery. In this case, since imaging in an operating room is scheduled, the control unit 107 displays the battery 120a on the display unit 114.

Next, a case is described where the radiation imaging apparatus 105 is scheduled to perform imaging in a visiting car in which there are many charging opportunities and the number of images to be captured from the completion of charging to until next charging is small. In this case, if the battery 120b is attached to the battery attachment unit 109, the control unit 107 performs display indicating that an appropriate battery is attached on the display unit 114.

Additionally, in a case where the identified battery 120 is a battery that is not registered in the table information in the control unit 107, the control unit 107 performs display indicating that the identified battery 120 is a battery that cannot be recognized on the display unit 114. Consequently, the control unit 107 informs the user of the radiation imaging apparatus 105 that an appropriate battery 120 should be attached.

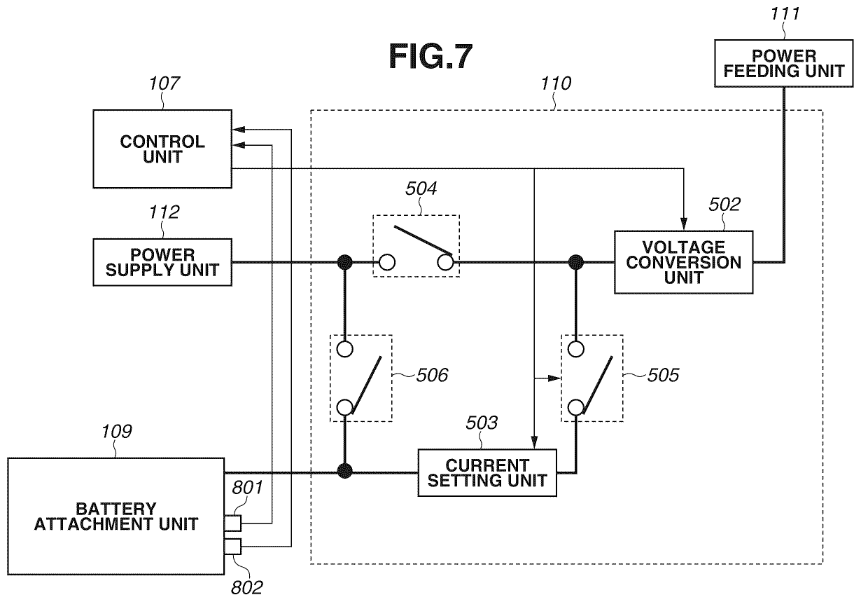

FIG. 7 is a diagram illustrating an example of the configuration of the charging circuit unit 110 in FIG. 6. The charging circuit unit 110 in FIG. 7 is different from the charging circuit unit 110 in FIG. 5 in that a switch 505 is added. The control unit 107 determines the type of the battery 120 attached to the battery attachment unit 109 based on output signals of the recessed portions 801 and 802 of the battery attachment unit 109. Similarly to the first exemplary embodiment, the control unit 107 controls the output voltage value of the voltage conversion unit 502, the output current value of the current setting unit 503, and the switch 505 according to the type of the battery 120. The switch 505 is controlled to be turned on only if power is to be fed from the power feeding unit 111 and the setting of the voltage of the voltage conversion unit 502 and the setting of the current of the current setting unit 503 are completed. Further, in the present exemplary embodiment, the voltage conversion unit 502 is configured to enable the selection of whether to execute voltage conversion. The voltage conversion unit 502 is configured to output the output power from the power feeding unit 111 as it is if voltage conversion is not to be executed.

A hot swapping function may be added to the switch 505. The addition of the hot swapping function to the switch 505 can reduce the influence of an inrush current when the output power of the voltage conversion unit 502 is input to the battery attachment unit 109.

When the control unit 107 sets the voltage conversion unit 502 and the current setting unit 503, a circuit that sets the output voltage of the voltage conversion unit 502 and sets the output current of the current setting unit 503 may be configured using a digital potentiometer.

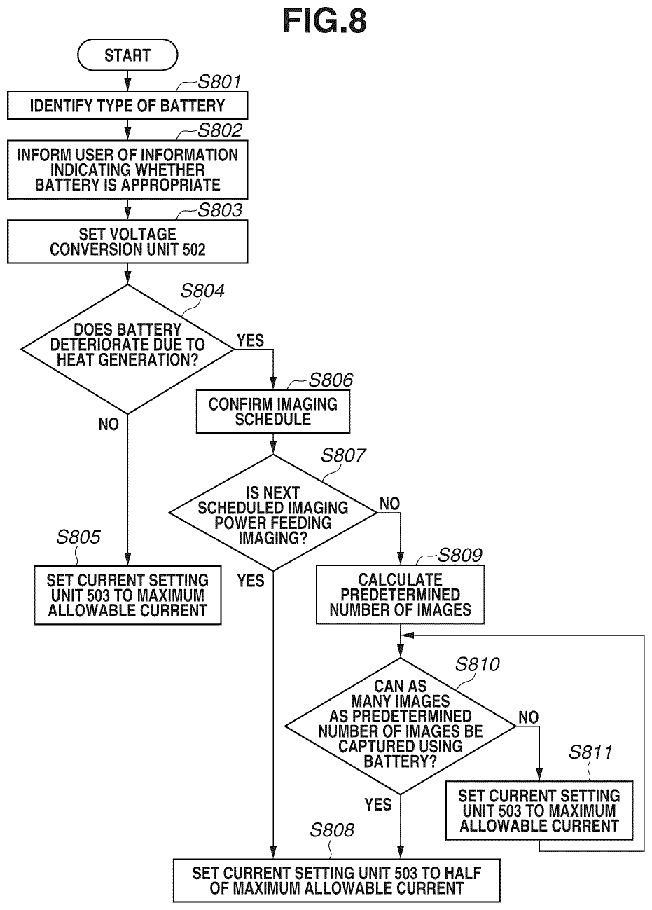

FIG. 8 is a flowchart illustrating an example of a charging method for charging the radiation imaging apparatus 105 in FIG. 6. With reference to FIG. 8, a description is given below of an operation for the control unit 107 to identify the battery 120 attached to the battery attachment unit 109 and set the charging circuit unit 110.

First, in step S801, the control unit 107 identifies the type of the battery 120 attached to the battery attachment unit 109. Specifically, the control unit 107 identifies the type of the battery 120 based on output signals of the recessed portions 801 and 802 of the battery attachment unit 109. Before identifying the type of the battery 120, the control unit 107 turns off the switch 505, thereby setting the voltage conversion unit 502 not to perform voltage conversion.

Next, in step S802, as described above, based on the identified type of the battery 120 and the imaging schedule information (the imaging use), the control unit 107 displays (informs the user of) information indicating whether the attached battery 120 is appropriate on the display unit 114.

Next, in step S803, the control unit 107 sets the output voltage of the voltage conversion unit 502 according to the type of the battery 120.

Next, in step S804, the control unit 107 determines whether the battery 120 attached to the battery attachment unit 109 is a battery that is to be subjected to soft charging in which heat generation due to charging is reduced by setting the charging current to be small. Specifically, if the lithium-ion battery 120a is attached, the control unit 107 determines that the lithium-ion battery 120a is a battery that is to be subjected to soft charging. If the attached battery 120 is a battery that is to be subjected to soft charging (YES in step S804), the processing proceeds to step S806.

Generally, if a lithium-ion battery or a lithium-ion polymer battery is used under high temperature, the reaction speed of a chemical reaction leading to deterioration accelerates. Thus, the progress of the deterioration of the battery becomes faster. Thus, if the battery is charged with the maximum charging current allowed by the battery to shorten the charging time, the deterioration becomes faster due to heat generation. If the battery deteriorates, the capacity of the battery decreases. Thus, the number of images that can be captured when the battery is fully charged decreases according to the deterioration. To decrease the progress speed of the deterioration, it is necessary to reduce the charging current and charge the battery with a small charging current.

If the attached battery 120 is not a battery that is to be subjected to soft charging (NO in step S804), the processing proceeds to step S805. Specifically, if the battery 120 attached to the battery attachment unit 109 is the lithium-ion capacitor battery 120b, the control unit 107 determines that the attached battery 120 is not a battery that is to be subjected to soft charging. Then, the processing proceeds to step S805. Generally, a lithium-ion capacitor battery uses a method for accumulating charges in the battery, not a chemical reaction, and therefore is less likely to deteriorate under high temperature. Thus, it is possible to charge the battery with the maximum charging current allowed by the battery without regard to deterioration due to heat generation.

In step S805, the control unit 107 sets the output current of the current setting unit 503 to the maximum allowable current of the battery 120 attached to the battery attachment unit 109. Then, the control unit 107 turns on the switch 505. The current setting unit 503 starts charging the battery 120 with the maximum allowable current of the battery 120.

In step S806, the control unit 107 checks the imaging schedule information received from the computer 103.

In step S807, the control unit 107 checks the imaging schedule information and determines whether the next scheduled imaging is power feeding imaging in which an image is captured in the state where the power feeding apparatus 130 is connected to the power feeding unit 111. If the next scheduled imaging is power feeding imaging (YES in step S807), the processing proceeds to step S808. If the next scheduled imaging is not power feeding imaging (NO in step S807), the processing proceeds to step S809. Regardless of whether the next scheduled imaging is power feeding imaging, the power feeding apparatus 130 is currently connected to the power feeding unit 111.

In step S809, based on the imaging schedule information, the control unit 107 calculates the number of images to be continuously captured from now by the feeding of power from the battery 120.

In step S810, the control unit 107 detects the remaining amount of the battery 120 attached to the battery attachment unit 109 and determines whether as many images as the number of images to be continuously captured from now by the feeding of power from the battery 120 can be captured. If as many images can be captured (YES in step S810), the processing proceeds to step S808. If as many images cannot be captured (NO in step S810), the processing proceeds to step S811.

In step S808, the control unit 107 sets the output current of the current setting unit 503 to half of the maximum allowable current of the battery 120 attached to the battery attachment unit 109. Then, the control unit 107 checks whether the switch 505 is on. If the switch 505 is off, the control unit 107 turns on the switch 505. The current setting unit 503 starts charging the battery 120 with half of the maximum allowable current of the battery 120.

In step S811, the control unit 107 sets the output current of the current setting unit 503 to the maximum allowable current of the battery 120 attached to the battery attachment unit 109. Then, the control unit 107 turns on the switch 505, and the processing returns to step S810. The current setting unit 503 starts charging the battery 120 with the maximum allowable current of the battery 120. The processing returns from step S811 to step S810, whereby the control unit 107 repeatedly determines whether as many images as the number of images to be continuously captured from now by the feeding of power from the battery 120 can be captured. The charging progresses, and the remaining amount of the battery 120 increases, whereby the processing proceeds from step S810 to step S808. In step S808, the current setting unit 503 changes the battery 120 to charging with half of the maximum allowable current of the battery 120.

In steps S805, S808, and S811, the charging circuit unit 110 starts constant current charging on the battery 120 with the output current set in the current setting unit 503. The charging circuit unit 110 performs constant current charging on the battery 120 with the output current set in the current setting unit 503 until the voltage in the battery 120 is equal to the output voltage set in the voltage conversion unit 502. Then, if the voltage in the battery 120 is equal to the output voltage set in the voltage conversion unit 502, the charging circuit unit 110 performs constant voltage charging on the battery 120 with the output voltage set in the voltage conversion unit 502.

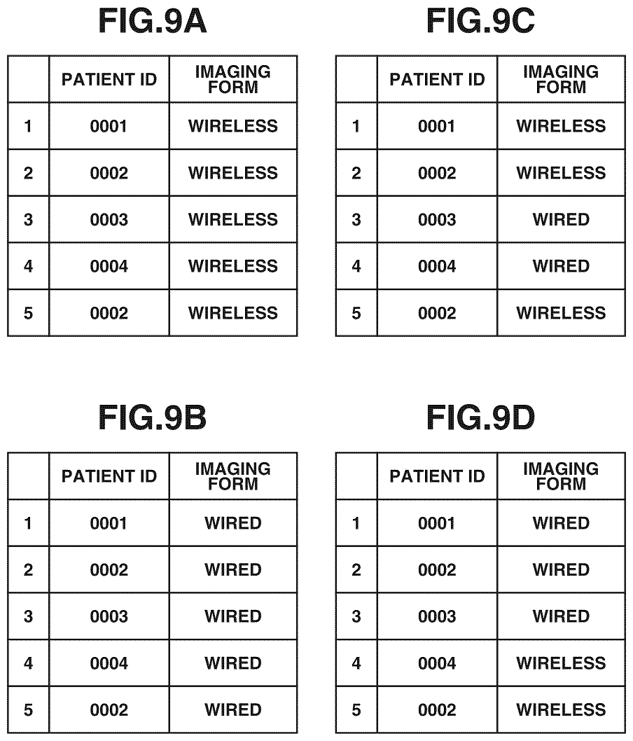

FIGS. 9A to 9D are diagrams illustrating examples of the calculation method in step S809 in FIG. 8. Based on the imaging schedule information, the control unit 107 calculates the number of images to be continuously captured from now by the feeding of power from the battery 120. Each of FIGS. 9A to 9D illustrates an example of a part of the imaging schedule information and illustrates an imaging order, a patient identifier (ID), and an imaging form. The imaging form indicates wired imaging in which an image is captured by wire or wireless imaging in which an image is captured wirelessly.

The imaging schedule information in FIG. 9A illustrates the state where five images are scheduled to be captured and the imaging forms of the images are all wireless imaging. In the case of wireless imaging, the radiation imaging apparatus 105 captures an image by the feeding of power from the battery 120 in the state where the power feeding apparatus 130 is not connected to the power feeding unit 111. In step S807, since the capturing of the first image as the next imaging is wireless imaging, the control unit 107 determines that the next imaging is not power feeding imaging. Then, the processing proceeds to step S809. In step S809, since five images will be captured in a row by wireless imaging by the feeding of power from the battery 120, the control unit 107 determines that the number of images to be continuously captured from now by the feeding of power from the battery 120 is five.

The imaging schedule information in FIG. 9B illustrates the state where five images are scheduled to be captured and the imaging forms of the images are all wired imaging. In the case of wired imaging, the radiation imaging apparatus 105 captures an image by the feeding of power from the power feeding apparatus 130 in the state where the power feeding apparatus 130 is connected to the power feeding unit 111. In step S807, since the capturing of the first image as the next imaging is wired imaging, the control unit 107 determines that the next imaging is power feeding imaging. Then, the processing proceeds to step S808. Since the next imaging is wired imaging, the number of images to be continuously captured from now by the feeding of power from the battery 120 is zero.

The imaging schedule information in FIG. 9C illustrates the state where five images are scheduled to be captured, the capturing of the first two images is wireless imaging, the capturing of the next two images is wired imaging, and the capturing of the last one image is wireless imaging. In step S807, since the capturing of the first image as the next imaging is wireless imaging, the control unit 107 determines that the next imaging is not power feeding imaging. Then, the processing proceeds to step S809. The capturing of the first two images is wireless imaging, and the capturing of the third image is wired imaging. Thus, in step S809, since two images will be captured in a row by wireless imaging by the feeding of power from the battery 120, the control unit 107 determines that the number of images to be continuously captured from now by the feeding of power from the battery 120 is two.

The imaging schedule information in FIG. 9D illustrates the state where five images are scheduled to be captured, the capturing of the first three images is wired imaging, and the capturing of the next two images is wireless imaging. In step S807, since the capturing of the first image as the next imaging is wired imaging, the control unit 107 determines that the next imaging is power feeding imaging. Then, the processing proceeds to step S808. Since the next imaging is wired imaging, the number of images to be continuously captured from now by the feeding of power from the battery 120 is zero.

As described above, in the radiation imaging apparatus 105, the batteries 120a to 120c can be selectively attached to the battery attachment unit 109. The charging circuit unit 110 charges the battery 120 with a current and a voltage according to the battery 120 attached to the battery attachment unit 109 and thereby can perform charging in which the deterioration of the battery 120 is reduced. The reduction in the deterioration of the battery 120 can reduce a decrease in the number of captured images when the battery 120 is fully charged due to the deterioration of the battery 120. Based on the imaging schedule information, the control unit 107 can determine whether the attached battery 120 is appropriate.

Although the control unit 107 identifies the type of the battery 120 based on output signals of the recessed portions 801 and 802 of the battery attachment unit 109, the present invention is not limited to this. For example, a sub-battery for supplying power to the control unit 107 may be provided, a barcode may be provided on a side surface of the battery 120, and a barcode reading apparatus may be prepared in the battery attachment unit 109. The control unit 107 may input an output signal of the barcode reading apparatus or read battery information from a memory in the battery 120 and identify the type of the battery 120. If the voltages of the batteries 120a to 120c are different from each other, the control unit 107 may identify the type of the battery 120 based on the voltage of the battery 120.

Although in step S808 in FIG. 8, the control unit 107 sets the output current of the current setting unit 503 to half of the maximum allowable current of the battery 120 attached to the battery attachment unit 109, the present invention is not limited to this. The output current of the current setting unit 503 may only need to be set to less than the maximum current of the battery 120. That is, this is for the purpose of reducing heat generation in the battery 120, and therefore, the amount of current does not matter.

In step S804, if the battery 120 is a battery that deteriorates due to heat generation, the control unit 107 determines that the battery 120 is a battery that is to be subjected to soft charging. If the control unit 107 determines that the battery 120 is a battery that is to be subjected to soft charging, the control unit 107 may lower the output voltage of the voltage conversion unit 502. Generally, if the full-charge state of a lithium-ion battery that deteriorates due to temperature is maintained, a high voltage state is maintained in a cell within the lithium-ion battery, and a chemical reaction leading to deterioration is promoted. The voltage conversion unit 502 can reduce the deterioration of the battery 120 by decreasing the voltage when the battery 120 is charged so that the battery 120 is not fully charged.

The first and second exemplary embodiments are not limited to the above configurations. A configuration may be employed in which if the voltage from the power feeding unit 111 and the voltage of the battery 120 attached to the battery attachment unit 109 are the same, the power feeding unit 111 and the power supply unit 112 are connected together and the power feeding unit 111 and the current setting unit 503 are connected together not via the voltage conversion unit 502. Voltage conversion by the voltage conversion unit 502 causes power loss due to the DC/DC converter. Specifically, the power loss includes loss due to the flowing of a current to an inductor or a metal-oxide-semiconductor field-effect transistor (MOSFET) included in the circuit of the DC/DC converter and loss due to the switching of the MOSFET. If the voltage from the power feeding unit 111 and the voltage of the battery 120 attached to the battery attachment unit 109 are the same, the power feeding unit 111 feeds power not via the voltage conversion unit 502, whereby it is possible to feed power by reducing the loss of power. Thus, it is possible to reduce the power of the power feeding apparatus 130.

As described above, the battery attachment unit 109 is a battery mounting unit. Any type of battery selected from among a plurality of types of batteries can be mounted on the battery attachment unit 109, and the battery can be attached to and detached from the battery attachment unit 109. The control unit 107 is an identification unit and identifies the type of the battery mounted on the battery attachment unit 109. The charging circuit unit 110 is a charging unit and charges the battery mounted on the battery attachment unit 109 using a charging method according to the type of the battery identified by the control unit 107. To the power supply unit 112, power is fed from the battery mounted on the battery attachment unit 109.

Specifically, the charging circuit unit 110 charges the battery mounted on the battery attachment unit 109 with a current or a voltage according to the type of the battery identified by the control unit 107. The charging circuit unit 110 identifies the type of the battery mounted on the battery attachment unit 109 according to the shape of the battery mounted on the battery attachment unit 109. Alternatively, the control unit 107 may identify the type of the battery mounted on the battery attachment unit 109 according to information stored in a memory of the battery mounted on the battery attachment unit 109. Alternatively, the charging circuit unit 110 may identify the type of the battery mounted on the battery attachment unit 109 by reading information (e.g., a barcode) described in the battery mounted on the battery attachment unit 109.

In step S802, the control unit 107 functions as an informing unit, and according to the imaging schedule, informs the user of whether the type of the battery mounted on the battery attachment unit 109 is appropriate. For example, in a case where imaging for use in which there are few charging opportunities and the capacity of a battery is required is scheduled, and the battery mounted on the battery attachment unit 109 is a lithium-ion battery or a lithium-ion polymer battery, the control unit 107 informs the user that the type of the battery mounted on the battery attachment unit 109 is appropriate. In a case where imaging in which many charging opportunities can be secured is scheduled, and the battery mounted on the battery attachment unit 109 is a lithium-ion capacitor battery, the control unit 107 informs the user that the type of the battery mounted on the battery attachment unit 109 is appropriate.

Specifically, in a case where imaging in an operating room or emergency imaging is scheduled, and the battery mounted on the battery attachment unit 109 is a lithium-ion battery or a lithium-ion polymer battery, the control unit 107 informs the user that the type of the battery mounted on the battery attachment unit 109 is appropriate. In a case where imaging in a visiting car or an imaging room is scheduled, and the battery mounted on the battery attachment unit 109 is a lithium-ion capacitor battery, the control unit 107 informs the user that the type of the battery mounted on the battery attachment unit 109 is appropriate.

In step S804, if the type of the battery mounted on the battery attachment unit 109 is a predetermined type, the processing proceeds to step S806. For example, if the type of the battery mounted on the battery attachment unit 109 is a first type, the processing proceeds to step S805. If the type of the battery mounted on the battery attachment unit 109 is a second type, the processing proceeds to step S806.

In step S807, if the next imaging is imaging in which the battery mounted on the battery attachment unit 109 is used, the processing proceeds to step S809. If the next imaging is imaging in which the battery mounted on the battery attachment unit 109 is not used, the processing proceeds to step S808. The charging circuit unit 110 charges the battery mounted on the battery attachment unit 109 with different currents or voltages between a case where the next imaging is imaging in which the battery mounted on the battery attachment unit 109 is used and a case where the next imaging is imaging in which the battery mounted on the battery attachment unit 109 is not used.

In step S810, the charging circuit unit 110 charges the battery mounted on the battery attachment unit 109 with different currents or voltages according to whether the remaining amount of the battery mounted on the battery attachment unit 109 is a remaining amount with which as many images as the number of images scheduled to be captured from the next time onward using the battery mounted on the battery attachment unit 109 can be captured.

The charging current or the charging voltage in step S808 is a current or a voltage in a case where the remaining amount of the battery mounted on the battery attachment unit 109 is a remaining amount with which as many images as the above number of images to be captured can be captured. The charging current or the charging voltage in step S811 is a current or a voltage in a case where the remaining amount of the battery mounted on the battery attachment unit 109 is not a remaining amount with which as many images as the above number of images to be captured cannot be captured. The charging current or the charging voltage in step S808 is smaller than the charging current or the charging voltage in step S811.

In steps S805 and S811, the charging circuit unit 110 charges the battery mounted on the battery attachment unit 109 with the maximum allowable current or the maximum allowable voltage of the battery mounted on the battery attachment unit 109. In step S808, the charging circuit unit 110 charges the battery mounted on the battery attachment unit 109 with a current less than the maximum allowable current of the battery mounted on the battery attachment unit 109 or a voltage less than the maximum allowable voltage of the battery mounted on the battery attachment unit 109.

For example, each of the above exemplary embodiments can also be achieved by a computer of the control unit 107 executing a program. A method for supplying the program to the computer, e.g., a computer-readable recording medium such as a Compact Disc Read-only Memory (CD-ROM) that records the program or a transmission medium such as the Internet via which the program is transmitted, can also be applied as an exemplary embodiment. The program can also be applied as an exemplary embodiment. The program, the recording medium, the transmission medium, and a program product are included in the category of the present invention. A combination easily imaginable from the first and second exemplary embodiments can also be included in the category of the present invention.

All the above exemplary embodiments merely illustrate specific examples for carrying out the present invention, and the technical scope of the present invention is not interpreted in a limited manner based on these exemplary embodiments. That is, the present invention can be carried out in various ways without departing from the technical idea or the main feature of the present invention.

The present invention is not limited to the above exemplary embodiments, and can be changed and modified in various ways without departing from the spirit and the scope of the present invention. Thus, the following claims are appended to publicize the scope of the present invention.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

According to the present invention, it is possible to mount any type of battery, and it is possible to charge a battery using a charging method according to the type of the battery without a dedicated cradle.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

1. A radiation imaging apparatus comprising:
a battery mounting unit on which any type of battery selected from among a plurality of types of batteries is mountable and to and from which the battery is attachable and detachable;
an identification unit configured to identify a type of a battery mounted on the battery mounting unit; and
a charging unit configured to charge the battery mounted on the battery mounting unit using a charging method according to the type of the battery identified by the identification unit,
wherein power is fed from the battery mounted on the battery mounting unit.

2. The radiation imaging apparatus according to claim 1, wherein the charging unit charges the battery mounted on the battery mounting unit with a current or a voltage according to the type of the battery identified by the identification unit.

3. The radiation imaging apparatus according to claim 1, wherein the identification unit identifies the type of the battery mounted on the battery mounting unit according to a shape of the battery mounted on the battery mounting unit.

4. The radiation imaging apparatus according to claim 1, wherein the identification unit identifies the type of the battery mounted on the battery mounting unit according to information stored in a memory of the battery mounted on the battery mounting unit.

5. The radiation imaging apparatus according to claim 1, wherein the identification unit identifies the type of the battery mounted on the battery mounting unit by reading information described on the battery mounted on the battery mounting unit.

6. The radiation imaging apparatus according to claim 1, wherein, at a joining portion with the battery mounted on the battery mounting unit, the identification unit generates a signal for identifying the type of the battery mounted on the battery mounting unit.

7. The radiation imaging apparatus according to claim 2, wherein the charging unit performs constant current charging on the battery mounted on the battery mounting unit with the current according to the type of the battery identified by the identification unit and then performs constant voltage charging on the battery mounted on the battery mounting unit with the voltage according to the type of the battery identified by the identification unit.

8. The radiation imaging apparatus according to claim 1, further comprising an informing unit configured to, according to an imaging schedule, inform a user of whether the type of the battery mounted on the battery mounting unit is appropriate.

9. The radiation imaging apparatus according to claim 8,
wherein, in a case where imaging for use in which there are few charging opportunities and a capacity of a battery is required is scheduled, and the battery mounted on the battery mounting unit is a lithium-ion battery or a lithium-ion polymer battery, the informing unit informs the user that the type of the battery mounted on the battery mounting unit is appropriate, and
wherein, in a case where imaging in which many charging opportunities can be secured is scheduled, and the battery mounted on the battery mounting unit is a lithium-ion capacitor battery, the informing unit informs the user that the type of the battery mounted on the battery mounting unit is appropriate.

10. The radiation imaging apparatus according to claim 8,
wherein, in a case where imaging in an operating room or emergency imaging is scheduled, and the battery mounted on the battery mounting unit is a lithium-ion battery or a lithium-ion polymer battery, the informing unit informs the user that the type of the battery mounted on the battery mounting unit is appropriate, and
wherein, in a case where imaging in a visiting car or an imaging room is scheduled, and the battery mounted on the battery mounting unit is a lithium-ion capacitor battery, the informing unit informs the user that the type of the battery mounted on the battery mounting unit is appropriate.

11. The radiation imaging apparatus according to claim 2, wherein the charging unit charges the battery mounted on the battery mounting unit with different currents or voltages according to whether next imaging is imaging in which the battery mounted on the battery mounting unit is used, and a remaining amount of the battery mounted on the battery mounting unit is a remaining amount with which as many images as the number of images scheduled to be captured from a next time onward using the battery mounted on the battery mounting unit can be captured.

12. The radiation imaging apparatus according to claim 11, wherein a current or a voltage in a case where the remaining amount of the battery mounted on the battery mounting unit is a remaining amount with which as many images as the number of images to be captured can be captured is smaller than a current or a voltage in a case where the remaining amount of the battery mounted on the battery mounting unit is not the remaining amount with which as many images as the number of images to be captured can be captured.

13. The radiation imaging apparatus according to claim 11, wherein the charging unit charges the battery mounted on the battery mounting unit with different currents or voltages according to whether the type of the battery mounted on the battery mounting unit is a predetermined type, and the next imaging is imaging in which the battery mounted on the battery mounting unit is used, and the remaining amount of the battery mounted on the battery mounting unit is a remaining amount with which as many images as the number of images scheduled to be captured from a next time onward using the battery mounted on the battery mounting unit can be captured.

14. The radiation imaging apparatus according to claim 2, wherein the charging unit charges the battery mounted on the battery mounting unit with different currents or voltages between a case where next imaging is imaging in which the battery mounted on the battery mounting unit is used and a case where the next imaging is imaging in which the battery mounted on the battery mounting unit is not used.

15. The radiation imaging apparatus according to claim 14,
wherein in a case where the next imaging is imaging in which the battery mounted on the battery mounting unit is used, the charging unit charges the battery mounted on the battery mounting unit with a maximum allowable current or a maximum allowable voltage of the battery mounted on the battery mounting unit, and
wherein in a case where the next imaging is imaging in which the battery mounted on the battery mounting unit is not used, the charging unit charges the battery mounted on the battery mounting unit with a current less than the maximum allowable current of the battery mounted on the battery mounting unit or a voltage less than the maximum allowable voltage of the battery mounted on the battery mounting unit.

16. The radiation imaging apparatus according to claim 2,
wherein, in a case where the type of the battery mounted on the battery mounting unit is a first type, the charging unit charges the battery mounted on the battery mounting unit with a maximum allowable current or a maximum allowable voltage of the battery mounted on the battery mounting unit, and
wherein, in a case where the type of the battery mounted on the battery mounting unit is a second type, the charging unit charges the battery mounted on the battery mounting unit with a current less than the maximum allowable current of the battery mounted on the battery mounting unit or a voltage less than the maximum allowable voltage of the battery mounted on the battery mounting unit or charges the battery mounted on the battery mounting unit with the maximum allowable current or the maximum allowable voltage of the battery mounted on the battery mounting unit.

17. The radiation imaging apparatus according to claim 2, wherein the charging unit includes a current control unit configured to charge the battery mounted on the battery mounting unit with the current according to the type of the battery identified by the identification unit.

18. The radiation imaging apparatus according to claim 2,
wherein the charging unit includes:
a current control unit configured to charge the battery mounted on the battery mounting unit with the current according to the type of the battery identified by the identification unit; and
a voltage control unit configured to charge the battery mounted on the battery mounting unit with the voltage according to the type of the battery identified by the identification unit.

19. A charging method for charging a radiation imaging apparatus including a battery mounting unit on which any of a plurality of types of batteries is mountable and to and from which the mounted battery is attachable and detachable, the charging method comprising:
identifying a type of a battery mounted on the battery mounting unit;
charging the battery mounted on the battery mounting unit using a charging method according to the identified type of the battery; and
feeding power from the battery mounted on the battery mounting unit.

20. A storage medium storing a program for causing a computer to execute:
in a battery mounting unit on which any of a plurality of types of batteries is mountable and to and from which the mounted battery is attachable and detachable, identifying a type of a battery mounted on the battery mounting unit;
charging the battery mounted on the battery mounting unit using a charging method according to the identified type of the battery; and
feeding power from the battery mounted on the battery mounting unit.

21. A radiation imaging apparatus comprising a battery mounting unit to which any of a plurality of types of batteries is mountable, the radiation imaging apparatus operating with power fed from the battery mounted on the battery mounting unit, wherein the mounted battery is attachable to and detachable from the battery mounting unit.
